SEQUENCE LISTING (8 sequences)

SEQ ID NO: 1 - DNA, 24 bp, Yeast
cgtgtatatagcgtggatggccag

SEQ ID NO: 2 - DNA, 16 bp, Yeast
gcaccaccaccagtag

SEQ ID NO: 3 - DNA, 24 bp, Homo sapiens
gcattgtcccagtctgttcccttc

SEQ ID NO: 4 - DNA, 31 bp, Homo sapiens
ccggcgctatgaccacaaccaccaagaactg

SEQ ID NO: 5 - DNA, 24 bp, Homo sapiens
agactgcctcctccgggaaccccc

SEQ ID NO: 6 - DNA, 22 bp, Homo sapiens
gctggatgagaacgccaatgtc

SEQ ID NO: 7 - DNA, 21 bp, Homo sapiens
cggggaagtcctggcaagtgg

SEQ ID NO: 8 - DNA, 24 bp, Homo sapiens
cacttccaaatgcagctgcgctct

(19) United States
(12) Patent Application Publication
(10) Pub. No.: US 2002/0160380 A1
(43) Pub. Date: Oct. 31, 2002

(54) Combinatorial libraries by recombination in yeast and analysis method

(76) Inventors: Gilles Truan, Ollainville (FR); Valerie Abecassis, Villebon-Sur-Yvette (FR); Denis Pompon, Gif-Sur-Yvette (FR)

Correspondence Address: HELLER EHRMAN WHITE & MCAULIFFE LLP, 1666 K STREET,NW, SUITE 300, WASHINGTON, DC, 20006, US

(21) Appl. No.: 09/959,519

(22) PCT Filed: Jun. 13, 2001
(86) PCT No.: PCT/FR01/01831

(30) Foreign Application Priority Data
Jun. 14, 2000 (FR) 0007555

(51) Int. Cl. C12Q 1/68; G01N 33/53; G01N 33/569; C12N 1/18; C12N 15/74

(52) U.S. Cl. 435/6; 435/7.31; 435/483; 435/254.2

(57) ABSTRACT

The present invention relates to a method for producing combinatorial functional expression libraries using a combinatorial library of nucleic acids belonging to the same gene family, comprising a step of cloning by recombination in yeast. The invention also relates to a method for producing functional mosaic proteins and for analyzing a combinatorial functional expression library, by determining a sequential footprint for each of the mosaic proteins of the library.

The present invention relates to a method for producing combinatorial functional expression libraries using a combinatorial library of nucleic acids belonging to the same gene family, comprising a step of cloning by recombination in yeast. The invention also relates to a method for producing functional mosaic proteins and for analyzing a combinatorial functional expression library, by determining a sequential footprint for each of the mosaic proteins of the library.

The diversity of protein functions may be viewed as the result of gene evolution through mutation, recombination and selection events (1, 2). Various techniques have been developed in order to attempt to reproduce, on a laboratory scale, the various steps of the processes of natural evolution. Conventional approaches of molecular evolution use steps of random mutation and recombination by polymerase chain amplification (PCR)(2-5). Molecular evolution is an approach which has been used with success in biotechnology for modifying protein functions (5-12) and to allow better understanding of the mechanisms of substrate recognition (13). Molecular evolution constitutes an effective approach for understanding the role of regions of sequences for the protein function when said sequences are not included in highly conserved regions, when the three-dimensional structure is not known or when no information is available from modelling techniques (29).

In order to carry out molecular evolution experiments or DNA-shuffling, a gene library is used which may be generated by mutagenesis of a single sequence (14) or which may consist of a group belonging to the same family or subfamily of genes (15). The ‘family-shuffling’ technique has been described as a means of accelerating the processes of evolution (16), which allows the emergence of unexpected activities or properties in the novel proteins generated (14). This technique has thus allowed the creation of enzymes with a combination of parental properties of interest (17, 18), having increased thermal stability (14) or having novel substrate specificities (19).

However, while family-shuffling makes it possible to obtain improvements which imitate, in vitro, the processes of evolution, the construction of random libraries of mosaic structures which are not biased toward the reassembly of mainly parental structures is still an essential point.

The difficulties in obtaining a homogenous library by family-shuffling greatly increase when the similarities between the starting sequences used decreases (30, 31). Thus, a relatively small number (of the order of 10%) of chimeras has frequently been described (Kikuchi describes 1% of chimeric structures for 2 genes having 84% identity at the protein level, using conventional DNA-shuffling techniques (32)).

Various techniques have been developed in order to decrease the content of parental structures, including the use of single-stranded DNA as the starting point for the shuffling (giving 14% of chimeric structures for 2 genes having 84% identity at the protein level (33)) or limited enzymatic fragmentations (32, 34) giving, themselves, much higher chimera contents. However, the latter method has the drawback that the enzymatically generated fragments are not random fragments, which induces a limitation in the number of novel gene structures which may thus be produced.

Other groups have used in vivo recombination in prokaryotic systems in order to obtain chimeras (30, 35, 36). These methods, however, have the drawback that the functional expression of proteins in E. coli is not always the most suitable when eukaryotic proteins are involved, in particular when multiprotein complexes, membrane-bound proteins or any protein requiring eukaryotic cellular machinery for its activity are involved. In particular, some eukaryotic proteins have posttranslational modifications (glycosylation, etc.) which cannot be carried out in prokaryotic hosts.

An object of the present invention is therefore to provide a method for constructing combinatorial functional expression libraries using nucleic acids belonging to the same gene family, which makes it possible to obtain libraries with the required complexity, i.e. with a large portion of the possible chimeric structures, and with a relatively low content of parental structures. Moreover, the method of the present invention makes it possible to obtain libraries which allow better expression of eukaryotic proteins.

The present invention also discloses a method for analyzing the gene sequences of a combinatorial library, in particular obtained using the method according to the invention, which makes it possible to associate a ‘footprint’ with each sequence variant present in said library. This analytical method makes it possible, in combination with a method for analyzing the functions and/or activities of the proteins of said library, to relate said sequence structures and said funtional structures. Thus, the combination of these two methods may be used to ‘pilot’ the mixing of genetic information, in order to obtain proteins of interest in a directed, more controlled, more rapid and less expensive way.

Thus, the present invention relates to a method for constructing a combinatorial functional expression library using a library of nucleic acids belonging to the same gene family, characterized in that it comprises the steps consisting in:

a. introducing said library of nucleic acids into a yeast, simultaneously with an expression vector,

b. obtaining said functional expression library by recombination of said combinatorial library of nucleic acids with said expression vector in said yeast.

A combinatorial functional expression library obtained using such a method according to the invention is also a subject of the invention.

Preferably, the expression vector with which the recombination is carried out in the yeast is linearized at the normal cDNA cloning site and has transcription promoter and termination sequences, the recombination being carried out at the level of said sequences.

The fragments of nucleic acids belonging to the library introduced into the yeast in step a. may or may not be fragmented. When these fragments are fragmented, this makes it possible to increase the in vivo recombination efficiency, which increases the diversity of the library since a recombination event must occur before the cloning into the expression vector. These points will be discussed later.

The recombination events taking place in the yeast may be homologous recombination events (between identical sequences) or homeologous recombination events (between sequences having a sufficient degree of identity).

The method according to the invention is also very advantageous in that it does not require a step involving passage in a prokaryote in order to obtain the combinatorial library.

Thus, the method according to the present invention allows a combinatorial expression library to be obtained directly in a eukaryotic host, which has a definite advantage for the expression of eukaryotic proteins, in particular membrane-bound proteins, or proteins belonging to multiprotein complexes.

The method according to the present invention therefore relates to a method for producing combinatorial libraries enhanced by recombination in yeast (CLERY).

Yeast (which may be modified at the genomic level) is also advantageously used as a tool for expression (39) of chimeric genes, which makes it possible to enhance the functional expression of the novel eukaryotic proteins obtained by this method (in particular the multiprotein complexes or the membrane-bound proteins). Moreover, the genomic modification of the strain of yeast used may make it possible to recreate the natural functioning environment (and therefore to optimize the screening possibilities), by producing other eukaryotic proteins essential for the activity of the novel proteins created, in particular in the case of multiprotein complexes.

The method according to the invention allows the final production of a combinatorial functional expression library by virtue of two different steps:

the cloning of the nucleic acid library into the expression vector simultaneously introduced into the yeast, by in vivo homologous recombination, makes it possible to obtain a functional expression library

the homologous or homeologous (between similar but not identical sequences) recombination which may occur in vivo in the ueast, between the various nucleic acids of the combinatorial library introduced into said yeast, makes it possible to increase the complexity and diversity of the combinatorial functional expression library obtained.

Thus, when the fragments of nucleic acids of the combinatorial library introduced into said yeast are fragmented and do not possess the two recombinogenic ends which allow cloning into the expression vector, it is essential for a recombination event to take place between two suitable fragments prior to said cloning.

Similarly, in a particular case of implementation of the method according to the invention, the production of at least one homeologous recombination event is observed in the library obtained, in particular due to the fact that the nucleic acids of the library initially introduced into the yeast belong to the same gene family.

For the purposes of the invention, the expression ‘nucleic acids belonging to the same gene family’ is intended to mean nucleic acids having a minimum of 35% identity, preferably 40%, more preferably 50%, or even 70%. These nucleic acids will be referred to as belonging to the same gene family if they have the above percentage identities, and may encode proteins having different activities and/or functions. These amino acids may encode proteins found naturally, or be ‘artificial’ nucleic acids, i.e. nucleic acids encoding proteins which are not found naturally. In particular, such ‘artificial’ nucleic acids encompass fusion proteins or proteins already obtained using DNA-shuffling methods.

For the purposes of the present invention, the term ‘percentage identity’ between two nucleic acid or amino acid sequences is intended to denote a percentage of identical nucleotides or of identical amino acid residues between the two sequences to be compared, obtained after the best alignment, this percentage being purely statistical and the differences between the two sequences being distributed randomly and throughout their length. The term ‘best alignment’ or ‘optimal alignment’ is intended to denote the alignment for which the percentage identity determined as below is highest. Sequence comparisons between two nucleic acid or amino acid sequences are conventionally carried out by comparing these sequences after having optimally aligned them, said comparison being carried out by segment or by ‘window of comparison’ in order to identify and compare local regions of sequence similarity. The optimal alignment of the sequences for the comparison can be carried out, other than manually, using the local homology algorithm of Smith and Waterman (49), using the local homology algorithm of Neddleman and Wunsch (50), using the similarity search method of Pearson and Lipman (51), using computer software which uses these algorithms (GAP, BESTFIT, BLAST P, BLAST N. FASTA and TFASTA in the Wisconsin Genetics Software Package, Genetics Computer Group, 575 Science Dr., Madison, Wis.). In order to obtain optimal alignment, the BLAST program is preferably used with the BLOSUM 62 matrix. The PAM or PAM250 matrixes may also be used.

The present invention thus makes it possible to obtain, with a high yield, recombinatorial libraries using nucleic acids with a much lower identity than the identity currently required in the state of the art (generally greater than 70%).

The nucleic acid library introduced into the yeast in step a. of the method according to the invention is preferably, itself, a combinatorial nucleic acid library.

This nucleic acid library is preferably a mixture of PCR products obtained by amplifying a combinatorial open reading frame library, using a pair of primers located in regions flanking said open reading frames. This combinatorial open reading frames library is obtained from sequence variant DNAs differing by one or more mutations and belonging to the same gene family for the purposes of the invention.

A single pair of primers is preferably used to carry out the PCR reaction as described in the paragraph above, but those skilled in the art may also use different pairs of primers. It is, however, more practical to use a single pair of primers.

In particular, a pair of primers is used which is located in translation termination and promoter regions in yeast, these being regions which allow the expression of open reading frames in this organism. Thus, it is likely that these regions, which will be present on all the DNA fragments of the nucleic acid library introduced into the yeast, will be the nucleic acid sequences involved in recombination with the sequences homologous to the expression vector cointroduced, which will allow the cloning of the open reading frames in said vector and the formation of the functional expression library.

As specified above, the nucleic acid library introduced into the yeast is preferably, itself, a combinatorial library of nucleic acids belonging to the same gene family for the purposes of the invention. This combinatorial library may be obtained using conventional methods of DNA fragmentation and reassembly by primer extension.

The DNA fragmentation step is carried out using methods known to those skilled in the art, such as for example digestion via restriction enzymes or nebulization. It is, however, preferred to fragment the DNA by partial digestion with a DNase, preferably DNaseI, which makes it possible to obtain fragments of a desired size in a more controlled way. Moreover, this makes it possible to effectively obtain random fragments, which is not always the case with the other enzymatic fragmentation techniques. In practice, and in order to obtain a combinatorial library with a great variety of combination and a large number of different mosaic proteins, the aim is to obtain fragments of a size between 15 and 700 base pairs (bp), preferably from 40 to 500 bp, more preferably from 100 to 300 bp.

The fragments are reassembled with one another using a primer extension technique. In principle, the fragments obtained are able to hybridize, and the addition of a DNA polymerase makes it possible to obtain extension of the hybridized fragments and reconstitution of functional genes, by several extension cycles.

Thus, a subject of the present invention is also a method for constructing a combinatorial functional expression library using a combinatorial library of nucleic acids belonging to the same gene family, comprising the steps consisting in:

a. introducing said combinatorial nucleic acid library into a yeast, simultaneously with an expression vector,

b. obtaining said functional expression library by recombination of said combinatorial nucleic acid library with said expression vector in said yeast,

the said combinatorial nucleic acid library being a mixture of PCR products obtained by amplifying a combinatorial open reading frame library, using a pair of primers located in regions flanking said open reading frames, said combinatorial library being obtained from homologous or sequence variant DNAs differing by one or more mutations, and said combinatorial open reading frame library being obtained by reassembly by “primer extension” of fragmentation products from at least two open reading frames encoding functional proteins, said open reading frames exhibiting more than 40% sequence identity with one another.

Those skilled in the art are aware of other techniques which allow recombination between DNA fragments and mixing thereof (DNA shuffling). Thus, an alternative method is the oligoligation method, which may optionally be used with heat-stable ligases. Other suitable methods may be chosen by those skilled in the art for the nucleic acid shuffling.

In order to assemble the fragments, a polymerase amplification reaction (PCR) is preferably used. The various steps of this reaction must be controled in order to be able to obtain a considerable amount of mosaic genes. Thus, the hybridization step is a very important step for ensuring the possibility of obtaining recombination between fragments exhibiting relatively low sequence identity, in particular for the low values of genes belonging to the same gene family (35% or 40%). Thus, the PCR reaction preferably carried out during the reassembly step is characterized in that each of its cycles has at least two hybridization stages, preferably at least four stages, with decreasing temperatures regularly spaced out. It is also important that the total duration of all of the hybridization steps is more than four minutes. One particular embodiment of the PCR reaction is such that each cycle has at least four hybridization stages of more than 60 seconds, with decreasing temperatures regularly spaced out.

The inventors have in fact shown that these reassembly conditions make it possible to obtain fragments of a size greater than the starting nucleic acids. In particular, when the starting nucleic acids are expression vectors carrying the genes of the same gene family, the fragmentation and reassembly steps may make it possible to obtain DNA fragments which are transformant in the yeast, i.e. which carry both the mosaic genes and the elements of the vector which allow it to replicate and to be maintained in the yeast. This ensures that the reassembly method according to the present method is extremely efficient (see also the examples).

In order to obtain a functional expression library in the yeast, the method according to the present invention proposes the co-introduction of an expression vector and of a library of nucleic acids belonging to the same gene family, which has been obtained by family shuffling as described in the above paragraphs.

In order to obtain said nucleic acid library, it is advantageous to start with nucleic acids belonging to the same gene family, which have already been cloned into an expression vector. Preferably, these nucleic acids are all cloned into the same expression vector and said vector is used for the co-introduction into the yeast.

Thus, after the reassembly step described above and since the conditions used make it possible to obtain long fragments, in particular of a size equal or greater than the size of the starting vector (i.e. longer than the nucleic acids belonging to the same gene family, the shuffling of which is the aim), a PCR reaction is carried out using a pair of primers located in the regions flanking the open reading frames. They are preferably primers located in the expression vector and they are chosen in particular in the transcription promoter and termination regions of said vector, as specified above.

Starting DNA which may thus be used is any vector containing the nucleic acids belonging to the same gene family, the recombination of which is desired. A vector which is multicopy in yeast, a vector which is single-copy in yeast or a vector for which the multi- or single-copy nature is unducible, may be chosen. An expression vector for a yeast, or an expression vector for a eukaryotic cell, which is a shuttle for yeast, may also be chosen. A vector which contains the elements required for autonomous replication in Escherichia coli may also be chosen. It is also, of course, possible to use a vector which has none of the properties developed above or which has a combination of said properties.

Preferably, the method according to the invention is carried out by choosing, as the starting vector, the expression vector co-introduced into the yeast with the nucleic acid library.

This expression vector has the elements for autonomously replicating in yeast as a multicopy vector, a single-copy vector or a conditional vector. It may also have genes which allow it to be selected on suitable media, in particular genes for resistance to antibiotics or for complementation of auxotrophy if the yeast used has this property.

The expression vector may be an expression vector for yeast. In this case, it has elements which allow effective transcription and translation in yeast. It may alternatively be a vector for expression in another host, which may be prokaryotic or eukaryotic, i.e. it may have the elements (origins of replication) allowing it to autonomously replicate in this other host. A vector which allows expression in a higher eukaryotic host, in particular a mammalian cell, is preferably chosen. Such a vector combines, with an expression cassette for a higher eukaryote, an origin of replication and a selection marker for yeast.

The vector preferably comprises a promoter, translation initiation and termination signals and also suitable transcription regulation regions. It may optionally have particular signals which specify secretion of the translated protein. The vectors which may be used are well known to those skilled in the art.

Use is preferably made, as a vector carrying the nucleic acids belonging to the same gene family, the fragmentation of which is desired, of a vector which has a size, including the open reading frames, greater than 7 kilobases (kb). The same vector may be used for the co-introduction into the yeast, for the step of recombination in the yeast.

The recombination is performed in yeast, preferably a yeast of the Saccharomyces genus, more preferably S. cerevisiae. It is, however, possible to use other types of yeast, among which Candida, Yarrovia, Kluyveromyces, Schizosaccharomyces, Torulopsis, Pichia and Hansenula. Those skilled in the art will choose the yeast depending on their competence and knowledge and on the desired objective. This yeast may be modified at the genomic level so as to express exogenous proteins, making it possible to complement the mosaic proteins, the generation of which is the aim.

The method according to the present invention has several advantages which will in particular become apparent in light of the examples. However, some of them may be summarized:

the method does not require passage in a prokaryotic host in order to obtain the library, which simplifies the manipulations to be caried out;

The method according to the invention makes it possible, in a single step, to clone, into the expression vector, the nucleic acid library introduced into the yeast and to increase the diversity by homologous or homeologous recombination between the various nucleic acids of the combinatorial library introduced into the yeast;

when the expression vector is multicopy, a mixture of products is obtained in the yeast, consisting of several copies of said vector, each having a different mosaic gene. Each yeast clone obtained therefore individually contains a library of mosaic genes, and this makes it possible to test the activities of the various proteins rapidly and efficiently;

when the expression vector can also replicate in E. coli, it is then possible to segregate the various plasmids by preparing the plasmid DNA of at least one yeast clone obtained, transforming E. coli with said extracted plasmid DNA and selecting the transformed clones on suitable medium so as to be able to descriminate between the elements of the combinatorial functional expression library.

Thus, those skilled in the art wishing to improve the functional properties of a protein may prepare, using the method according to the invention, a combinatorial functional expression library in yeast using nucleic acids of interest belonging to the same gene family. They may then test the yeast clones in order to select those for which the desired property is apparent, and obtain the truly advantageous sequences by performing the discrimination by passage in a prokaryotic host.

The method according to the invention thus makes it possible to produce functional active mosaic proteins, which are themselves subjects of the invention. Thus, a subject of the invention is also a method for producing functional active mosaic proteins, characterized in that a combinatorial functional expression library is constructed using a method according to the invention, in that the mosaic proteins are expressed and in that the functional active mosaic proteins are selected by studying their activity.

Preferably, the mosaic proteins, the generation of which is the aim, are enzymes having enhanced activities (heat-stability, novel function, modification of function, increase in activity, modification of substrate specificity, modification of activity in a precise environment, such as solvent, a pH, etc.). The use of the method according to the invention in order to generate novel enzymes has many advantages, since the activities of the novel proteins generated can then often be tested directly in the yeast. Starting nucleic acids which are then preferably used are nucleic acids belonging to the same gene family, which encode enzymes. The active mosaic proteins obtained are then termed derived from enzymes.

The examples of the present invention show the use of the method in generating novel proteins derived from cytochrome P450s. The cytochrome P450s (P450s) can recognize a wide variety of substrates and catalyze an even greater number of reactions. These enzymes have been demonstrated in practically all living organisms (20). In mammals, the P450s are involved in the formation of steroid hormones, but also have a predominant role in the metabolism of medicinal products and of polluants which can sometimes lead to processes of chemical carcinogenesis and toxicity (20-22). The human P450s 1A1 and 1A2 exhibit about 70% sequence identity and have certain different substrate specificities. They are among the P450s which are the most active in the metabolism of chemical carcinogens (23) and are involved, in humans, in lung cancer, for CYP1A1 (24-26), and in the activation of promutagens contained in food (27) or in liver cancers induced by aflatoxin B1, for CYP1A2. All of the properties of mammalian P450s in fact make them excellent candidates for the use of these techniques of molecular evolution (28).

A particular case of the present invention therefore relates to the method according to the present invention, also characterized in that the eukaryotic expression vector used for the shuffling contains an open reading frame encoding a eukaryotic membrane-bound enzyme. Preferably, said eukaryotic enzyme is chosen from the group consisting of eukaryotic cytochrome P450s, eukaryotic conjugation enzymes (phase II enzymes) and members of the eukaryotic ABC transporter family.

In this case, it may be advantageous to use a yeast strain which has a genetic modification allowing the overexpression of at least one protein chosen from the group consisting of an endogenous or exogenous P450 reductase, an adrenodoxin, an adrenodoxin reductase, a heterologous cytochrome b5 and a phase II enzyme (in particular an epoxide hydrolase). Such strains are described in patent EP 595 948. These strains in particular make it possible makes it possible to recreate the natural environment for the functioning of eukaryotic P450s (40,41).

The use of genetically modified yeast strains also makes it possible to recreate protein complexes with several fixed elements (elements expressed constitutively by the yeast) and a variable element (the product of the mosaic genes obtained using the method according to the invention).

The method according to the present invention can also be applied to other proteins. For example, it may be advantageous to generate receptors, which makes it possible to determine the sequences involved in the recognition and combination of the ligand, or chimeric proteins based on the proteins which are targets for antibiotics, which make it possible to determine the degrees of resistance as a function of the mutations.

It is usually necessary to carry out many “DNA-shuffling” cycles before obtaining a protein having the desired characteristics and/or properties. In the present case, after selecting the yeast clones expressing proteins having an activity close to the desired activity, it is possible to carry out a simple PCR reaction directly on said clones, using suitable primers flanking the open reading frames, and to carry out further shuffling by repeating the steps of the method according to the invention.

It is, however, desirable to be able to improve the rapidity with which the desired properties are obtained, by producing a relationship between the sequence structures of the mosaic proteins obtained and the functional structures of said proteins. This then makes it possible to easily relate the DNA sequences of the gene, or the links between the sequences, to an enzymatic function or another function (attachment of a substrate, thermophilicity, etc.).

The present invention therefore also relates to a method for analyzing a combinatorial functional expression library, characterized in that it comprises the following steps:

a. transformation of an Escherichia coli strain with the plasmid DNA extracted from the yeast strain or from a pool of yeasts,

b. hybridization of the plasmid DNA contained in each of the individual Escherichia coli clones obtained at the end of step a. with one or more probe(s) specific for a parental sequence.

This method, improved with steps which will subsequently be described, can be used on any combinatorial library, provided that there has been discrimination between the various nucleic acids forming the library.

The hybridization takes place on a DNA macro- or microarray, said array consisting either of the plasmid DNA contained in each of the individual Escherichia coli clones obtained at the end of step a., or of a PCR product thereof, or of said specific probes, attached to a solid support, each of the nucleic acids being located via its position in said array.

In the first case, the plasmid DNA contained in each of the individual Escherichia coli clones obtained at the end of step a., or a PCR product thereof, is attached to a solid support (glass, silicon, suitable membrane (nylon, nitrocellulose), etc.). The methods for attaching the DNA are known to those skilled in the art and the DNA can be fixed more or less solidly to the support used. It is not always necessary to extract the plasmid DNA from the E. coli clones obtained, it being possible to lyse them directly on the solid support used, or it being possible to carry out the PCR for amplifying the fragments corresponding to the mosaic genes directly on the bacterial clones without prior DNA extraction.

In the second case, the probes are attached to the solid support. There are several methods for preparing a support bearing probes. The probes can be synthesized and then attached to the support (the arranging possibly occurring mechanically, electronically, by inkjet, etc.) or the probes can be synthesized directly on the support (by photochemical arrangement or by inkjet, for example). Those skilled in the art will choose the method which is most suitable for the desired result.

Depending on the number of probes used, a more or less fine hybridization footprint is obtained for each of the clones tested. The higher the number of probes, the finer the footprint obtained. Probes which are located homogeneously over the entire length of the gene may be chosen. Alternatively, it may be profitable to use probes which are targeted in a set of sequence regions which are known to encode regions which are important for the function and/or activity of the protein. Thus, a targeted sequence footprint can be obtained.

Moreover, the conditions for hybridizing the probes vary depending on the degree of specificity of said probes for each parental structure. Thus, when two parental structures differ by a single base on the fragment corresponding to the probe, it is necessary to apply stringency conditions which are higher than if the parental structures are very different. Those skilled in the art know how to determine the best hybridization conditions, in particular by following the teaching of Sambrook et al. It is also important to note that certain mosaic genes may exhibit a weaker strength of hybridization with a given probe than other genes. Specifically, the effectiveness of the transfer of the DNA onto the solid support may have occurred more or less effectively, or the region of the gene to which the probe should hybridize is, itself, mosaic and consists of fragments originating from different “parent” genes.

A statistical analysis of the hybridization strengths may then be carried out, using a suitable computer program. The program first converts the hybridization signals into data of a parental type using a mask system with an XOR Boolean function, before the statistical analysis per se.

The analysis of the combinatorial library may take place in the following way:

A code is attributed to each nucleic acid sequence generated, depending on the capacity of the probes used to hybridize said sequence. It may be advantageous to use binary coding (0 if the site probed corresponds to a certain parental type, 1 if it corresponds to the other parental type), but other types of coding may also be used. Thus, each sequence generated in the library has an individual “signature”. When 6 probes are used and binary coding is used, 2⁶ possibilities are envisioned (from 000000 to 111111).

The frequency of each of the signatures thus obtained is then compared with the frequency expected if the DNA shuffling was occurring entirely randomly (in the case of 6 probes, the theoretical frequency of each pattern is then ½⁶). This analysis makes it possible to define a “preferential parent” for each of the positions probed (certain corrections must sometimes be made, in particular when the proportions of starting parental nucleic acids are not equal).

Studying the signatures also makes it possible to specify the relationships which may exist within the same mosaic, in particular the combinations between parental types, which may be found between each segment. For example, it is important to be able to easily determine the need for a correlation between two nucleic acid segments which are not necessarily adjacent in order to obtain a biological function.

The analysis may also be refined in order to obtain results which may provide several pieces of information. The examples illustrate such a step in disclosing a method in which each signature of the library is converted into a decimal number and in which a curve, which bears said decimal number on the x-axis and the cumulative frequency on the y-axis, is plotted. The analysis of said curve, and the modeling thereof by simulation, also make it possible to obtain valuable information concerning the probability of obtaining a certain type of parental structure at a given site, and the correlations existing between various fragments.

The statistical analyses thus described are facilitated by using computer tools, the development of which does not pose any problem to those skilled in the art.

The simulations of correlations between various segments may be produced by generating grids which are more or less random depending on the desired correlations. For example a grid may be generated for which a segment has more than a 50% probability of being of the same parental type as the neighboring segment. The number of grids which can thus be generated is extremely large and can thus make it possible to define an approximation of the results observed.

When correlations are observed between various segments, it is probable that applying a functional selection to the population of clones (which thus decreases the population of sequences which pass the screen) will lead to an increase in the number of correlations and to an evolution (convergence) of the statistical results obtained. The appearance of a pattern characteristic of the selection applied should therefore be obtained, which gives a sequence signature dependent on the functional selection applied to the system.

In summary, the present invention also relates to a method for analyzing hybridization footprints which can be obtained using the method for analyzing the combinatorial library described above, characterized in that it comprises the steps consisting in:

a. calculating the frequency of appearance of each of the possible combinations,

b. defining a signature of the statistical distribution of the combinations, using suitable mathematical and statistical processing.

Thus the present invention provides a means of very effectively producing combinatorial functional expression libraries using nucleic acids belonging to the same gene family for the purposes of the invention, which may have a relatively low degree of identity.

Moreover, the present invention has the advantage that it is possible to carry out the activity assay for the mosaic proteins produced, directly on the yeast clones obtained, without a prior purification step.

The present invention also provides a method for analyzing combinatorial libraries, based on hybridization and statistical analysis of the hybridization footprints obtained.

The present invention therefore provides tools which may be used for determining the links which may exist between the sequence structures and the functional structures of proteins. Thus, the present invention also relates to a method for determining links between sequence signatures and functional signatures of a protein, characterized in that it comprises the steps consisting in

a. preparing a combinatorial functional expression library using a method according to the invention,

b. producing the functional active mosaic proteins,

c. analyzing the functional differences and/or the differences in activity between said mosaic proteins,

d. analyzing the nucleic acids corresponding to said mosaic proteins using a method of analysis by hybridization according to the invention, optionally followed by statistical analysis using a method according to the invention,

e. relating the differences in sequence structure observed in step d. with the functional differences and/or the differences in activity observed in step c.

The implementation of this method, for identifying the important sequence regions or the links between sequence regions related to a function of interest, makes it possible to predict the structures which have said function, by deducing the structure being sought, as a function of the structure-function relationship obtained using the method described above.

Thus, it becomes possible to obtain proteins which have enhanced properties, as described above, or proteins which recognize a large number of substrates (“generic” enzymes), by piloting the mixing of genetic information in order to obtain the proteins of interest more rapidly and more effectively.

The various methods described in the state of the art made it possible to obtain the proteins of interest by repeating the DNA shuffling, subjecting the proteins obtained to increasingly fine screens. The present invention, which makes it possible to relate the structures and functions of the mosaic proteins obtained, makes it possible to carry out further DNA shuffling using, as starting nucleic acids, only the nucleic acids which have been identified as bearing the structures or structural organizations of interest.

Thus, the present invention relates to a method for obtaining a protein having enhanced properties, characterized in that it comprises the steps consisting in:

a. constructing a combinatorial functional expression library using a method according to the invention,

b. analyzing said combinatorial functional expression library,

c. analyzing the hybridization footprints obtained in step b. using a method according to the invention,

d. determining the links between the between sequence structures and functional structures of the proteins by comparing said hybridization footprints with the properties of the corresponding mosaic proteins,

e. predicting the structures of interest or the structural organizations in the mosaic proteins,

f. repeating steps a. to e., using, as starting nucleic acids for generating the combinatorial functional expression library, the nucleic acids bearing the structures of interest or the structural organizations identified in step e., a sufficient number of times to obtain the protein having desired enhanced properties.

Step f. consists in repeating the preceding steps until it has been possible to identify a protein having the desired properties. The present invention should make it possible to decrease the number of cycles for producing a combinatorial library/analyzing the proteins, compared to the methods of the prior art.

The proteins obtained using the method described are also a subject of the invention.

The invention also relates to a method for determining a protein structure which is important in response to a selection pressure, using a combinatorial functional expression library which has been obtained using a method according to the invention, and for the elements of which a signature has been obtained, comprising the steps of:

normalizing said library, by making the signatures homogeneous, for example by sorting using a suitable robotic machine. This step makes it possible to ensure that each footprint has the same probability in the normalized library,

applying a selection pressure,

analyzing the resulting expression library but using the methods for analyzing a sequence signature according to the invention,

studying the changes in sequence signatures induced by the selection pressure on the initial normalized library and deducing therefrom the structures selected or counter-selected in response to the selection pressure.

It should be noted that normalizing the library before applying the selection pressure in fact makes it possible to screen a greater diversity while screening the same number of clones as would be the case if there had been no normalization. Specifically, it may be observed that certain structures (as analyzed by the footprints) are present with probabilities greater than would be expected in the case of random shuffling. The normalization therefore makes it possible to decrease the influence of this problem.

The following examples are limited to the generation of novel cytochrome P450s, in order to illustrate the invention. However, they should not be considered to limit the invention, and in particular the type of protein and nucleic acid which may be used in the methods described in the present invention. Those skilled in the art can thus easily implement the methods of the invention, substituting other genes for the cytochrome P450 genes described in the examples.

### DESCRIPTION OF THE FIGURES

FIG. 1: principle of the construction of the libraries. A: lane 1, DNA marker (λ DNA digested by Pst I); lanes 2, 3, 4 and 5, 6, 7 correspond, respectively, to the plasmids p1A1/V60 and p1A2/V60 digested with DNAse I. Lanes 2 and 5 correspond to the fragmentation with 0.0112 units, lanes 3 and 6 with 0.0056 units and lanes 4 and 7 with 0.0028 units of DNase I per μg of DNA. B: reassembly reaction. Lane 1, DNA marker; lanes 2, 3 and 4 correspond to the reassembly reactions between fragments of p1A1/V60 and p1A2/V60 when mixing, respectively, the reactions of lanes 2 and 5, 3 and 6, and 4 and 7. C: amplification reaction. Lane 1, DNA marker; lanes 2, 3 and 4 correspond, respectively, to the amplification with the plasmids PYeDP60, p1A1/V60 and p1A2/V60; lanes 5, 6 and 7 correspond to the amplification using the DNA reassembled beforehand as matrix (B2, B3 and B4). The band shown in lane 6, panel C, was purified and used, without modification, to cotransform S. cerevisiae with the plasmid pYeDP60 linearized beforehand. The existence of recombination events between the various nucleic acids of the library introduced into the yeast is observed.

FIG. 2: Respective positions and sequences of the six probes used to produce the library characterization matrices. The numbers along the top or along the bottom correspond to the 5′ position for alignment of each probe on the sequences. The probes along the top and the bottom hybridize the sequences of P450 1A1 or of P450 1A2, respectively. The vertical bars in the central rectangle represent all the positions of mismatch between the sequence of P450 1A1 and of P450 1A2.

FIG. 3: The hybridization results were processed in Microsoft Excel, generating a 384-point grid with the following color code: the dark squares represent structures assimilated to structures of parental type (1A1 or 1A2) for the sequence regions corresponding to the six probes and the light squares represent mosaic structures.

FIG. 4: Experimental and theoretical cumulative frequencies for the observation of the 64 possible types of mosaic structure. The horizontal axis corresponds to coding for the mosaic structures using N=P1 +2*P2+4*P3+8*P4+16*P5+32*P6, in which P1 to P6 have the values of 0 or 1 depending, respectively, on the hybridization with the 1A1 or 1A2 sequences. The open circles represent the experimental curves deduced from the hybridization states of the 384-clone grid, with the six oligonucleotide probes. The continuous curve corresponds to theoretical curves when considering there to be a homogeneous proportion of 0.56:0.44 for the parental sequences 1A2 and 1A1 parental sequences and total shuffling (absence of cross-correlation). The broken-line curve represents the same curve for a proportion of 50:50 for the 1A1 and 1A2 parental sequences. The black circles represent the theoretical curve obtained with simulations when considering there to be a homogeneous proportion of 0.56:0.44 for the 1A2 and 1A1 parental sequences but a parental link probability of 0.1:0.6:0.85:0.1:0.1 between the 1-2, 2-3, 3-4, 4-5 and 5-6 probed segments, respectively. The link is defined as follows: 0 corresponds to independence and 1 corresponds to complete link.

FIG. 5: Representation of the parental and recombinant frequencies for the combination between two probes. The frequency of each combination was determined using one of the macros generated in Microsoft Excel. The sum of the four different frequencies (parental and recombinant) is always 1. A: combination between two adjacent probes; B: combination between probes separated by one probe; C: combination between distant probes (separated by two or three probes). The black and dark gray histograms represent the parental combinations while the light gray and the semi-dark gray represent the recombinant combinations.

FIG. 6: Colorimetric detection of mosaic structures functionally competent for naphthalene oxidation. The bioconversion is carried out in 1 ml of yeast culture in the presence of 1.6 mM naphthalene. The solid phase extraction and the development of the coloration are entirely carried out in microtitration plates as described in the examples. Dark coloration indicates positive clones.

FIG. 7 Diagrammatic representation of the sequences of 10 randomly selected mosaic structures: A in the total population; B: in the subpopulation of active clones. A nucleotide alignment with the two parental sequences was produced for each structure. These alignments were used as starting data for a sequence analysis program and a visualization program which generated the figure. The gray and black regions correspond, respectively, to sequences belonging to the 1A1 or 1A2 parental P450s. The upper or lower thin vertical lines indicate the regions of nucleotide mismatch with the second parental structure. The marks which cross the sequences indicate the positions of sequences which do not match with either of the two parental sequences and which must therefore correspond to mutations. The transparent horizontal portions correspond to segments of sequences for which it was not possible to determine, by sequence analysis, whether they belong to one or other of the parental types.

### EXAMPLES

### Example 1

Methods

1.A: Strains, Plasmids and Molecular Biology

Two S. cerevisiae strains were used: W303-1B, also named W(N) (Mat a; ade2-1; his3, leu2, ura3, trp1, can^{R}, cyr⁺), and W(R) which derives from W(N) by the insertion of the GAL10-CYC1 inducible promoter upstream of the endogenous yeast P450 reductase (YRED). This strain has been previously described by Truan et al. (40) and in patent EP 595 948, incorporated herein by way of reference.

The E. coli strain used was DH5-1 (F⁻, recA1, gyrA96, thi-1, hisR17, supE44, λ⁻) . The expression vectors used were p1A1/V60 (42) and p1A2/V60 (43, incorporated herein by reference); these two vectors were constructed by inserting the human CYP1A1 and CYP1A2 ORFs between the BamHI/KpnI and BamHI/EcoRI restriction sites, respectively, of pYeDP60. These two expression vectors also contain URA3 and ADE2 as selection markers and place the open reading frames (ORFs) under the control of the GAL10-CYC1 promoter and of the PGK terminator (39, incorporated herein by way of reference) . All the media used have been previously described in documents incorporated herein by way of reference (40, 42).

The DH5-1 bacteria were rendered electrocompetent according to the protocol described by Sambrook et al. (44) incorporated herein by way of reference, and the cells were transformed by following the recommendations of the manufacturer of the electroporator (Biorad). These cells were selected on solid LB media containing 50 μg/ml of ampicillin.

Transformation of Yeast

After preculturing for 12 hours in 5 ml of YPGA medium (for the W(N) strain) or YPLA medium (for the W(R) strain), the cells were diluted in 50 ml of YPGA medium so as to obtain a final density of 2×10⁶ cells/ml. Six hours later, the cells were washed twice with sterile water and once with TE-lithium acetate buffer (10 mM Tris-HCl, pH 7.5, 1 mM EDTA, 100 mM lithium acetate). The cells are then resuspended in 1 ml of TE-lithium acetate buffer.

The transformant DNA was then added to 50 μl of the previously obtained solution of cells, as were 50 μg of salmon sperm DNA (sonicated and denatured at 95° C., beforehand) and 350 μl of a 40% (w/v) solution of PEG 4000. This solution was then incubated at 30° C. for 30 minutes and subjected to a heat shock at 42° C. for 45 minutes. After centrifugation, the supernatant was removed and the cells were resuspended in 200 μl of a 0.1 M NaCl solution. The cells were then selected on a solid SWA6 medium (39, 42, incorporated herein by reference).

Extraction of the Plasmid DNA from Yeast

The colonies are resuspended in 1 ml of buffer A containing 2%o (v/v) of triton X-100, 50 mM of Tris-HCl, pH 8.0, 50 mM of EDTA and 200 mM of NaCl. Then, 1 volume of glass beads (Braun Scientifics, 0.45 mm diameter) was added and the solution was vortexed vigorously for 2 min with 300 μl of a phenol/chloroform/isoamyl alcohol (50:49:1, by vol.) mixture. After recovering the aqueous phase, the DNA was precipitated with ethanol and resuspended in 50 μl of water.

Sequences

Five bacterial clones derived from the initial library and five functional clones were randomly selected and sequenced. The sequences were produced either by ESGS (ESGS, group Cybergene, Evry France) or using the ABI kit and the ABI sequencer according to the manufacturer's (Perkin Elmer) protocols.

1.B: DNA Shuffling Based on Modified PCR

The technique used is derived from that described by Stemmer (2, 3, 15), incorporated herein by way of reference. The random fragmentation with DNase I (Grade II, Sigma-Aldrich) in the presence of Mn²⁺ is carried out with the modifications described by Lorimer and Pastan (45) and Zhao (46), incorporated herein by way of reference.

2.5 μg of each plasmid DNA (P1A1/V60 and P1A2/V60) were resuspended separately in a buffer containing 50 mM of Tris-HCl, pH 7.4, and 10 mM of MnCl₂ for a final volume of 40 μl. The DNase I was added at three different concentrations (0.0112 U/μg of DNA, 0.0056 U/μg of DNA and 0.0028 U/μg of DNA). The digestion was carried out at 20° C. for 10 min and the DNAse I was inactivated by heating at 90° C. for 10 min. The fragments obtained were purified on a Centrisep column (Princeton Separation Inc., Philadelphia, N.J.).

During the reassembly reaction, the purified fragments (10 μl of each fragmented plasmid) were amplified with a PCR reaction in 40 μl, using 2.5 U of Taq polymerase (Stratagene).

The PCR program used consisted of: 1 cycle of denaturation at 96° C., for 1.5 min; 35 cycles of (30s of denaturation at 94° C., 9 different hybridization steps each separated by 3° C., ranging from 65° C. to 41° C., and of 1.5 min and one elongation step of 1.5 min at 72° C.) and finally 7 min at 72° C.

The second amplification reaction was carried out with a 5′ primer located in the GAL10-CYC1 promoter (SEQ ID No. 1) and a 3′ primer located in the PGK terminator (SEQ ID No. 2).

1.C: Construction and Characterization of the Library

The PCR amplification products were separated by electrophoresis gel and then purified. The DNAs were inserted into pYeDP60 using in vivo recombination (gap repair) in yeast (37, 38, 43, 47, 48). The W303-1B strain was cotransformed with 1/20^{th} of the PCR product (insert) and 0.025 μg of pYeDP60 linearized beforehand using the EcoRI and BamHI restriction enzymes.

The DNA extracted from the yeast was used to transform the DH5-l strain of E. coli using the ampicillin resistance provided by the plasmid. 378 wells of a 384-well microtitration plate were inoculated with independent bacterial colonies chosen randomly from the library, 3 wells were inoculated with DH5-1 bacteria transformed beforehand with p1A1/V60 and the remaining 3 wells were inoculated with DH5-1 transformed with p1A2/V60. After 24 hours of growth in TB medium (44) containing 100 μg/ml of ampicillin, the 384 wells were then replicated on six Nylon N+ membranes (Amersham). Each filter was placed on a solid LB medium containing 100 μg/ml of ampicillin. After 12 hours of growth, the lysis of the bacterial colonies, the fixing and denaturation of the DNA and the prehybridization of the filters were carried out according to the protocol recommended by the manufacturer (Amersham).

11 pmol of oligonucleotides were added to 3.3 pmol of ³²P-labeled γ-ATP, 2 μl of polynucleotide kinase and 18 μl of buffer (New England Biolabs). The mixture was incubated for 2 h at room temperature. The filters were prehybridized according to the protocol recommended by the manufacturer. The labeled probe is added to a hybridization tube containing one of the filters and the whole is incubated for 12 h at 42° C. The filters are then washed in a solution of 2× SSPE/0.1% SDS for 10 min. The filters were analyzed by autoradiography, according to a known protocol.

Each probe was labeled a second time and hybridized to a different filter in order to be sure that the results were reproducible.

1.D: Selection of the Clones Containing Functional P450s

The bacterial colonies grew for 24 hours in 96-well microtitration plates. The DNA extraction was carried out using the protocol of the Multiscreen apparatus for mini-preparation of DNA by filtration in 96-well microplates (Millipore). Each purified DNA was used to transform the W(R) yeast strain in a 96-well microtitration plate and the cells were selected on solid SWA6 media.

After 3 days of growth at 30° C., 1 ml of SWA5 liquid medium was seeded with an aliquot of each colony, in a 96-well Deepwell microplate (ABGene) for 15 hours. The medium was then removed and replaced with 1 ml of YPLA medium containing 1.6 mM of naphthalene (Merck).

For each culture, the culture medium was then placed in the corresponding wells of a 96-well Multiscreen microplate (MABV N12, Millipore) containing 90 μl of functionalized octadecyl C18 silica gel resin (Aldrich). After filtration of the culture medium under vacuum, the substrate and the reaction products are bound to the silica. The resin was then washed twice with water and the metabolites eluted with 50 μl of isopropanol. After adding 20 μl of a 2 mg/ml solution of Diazo-Blue-B (Fluka), the colored reaction generated by the coupling between the diazo precursors and the phenols extracted from the culture medium was observed.

1. E: Statistical Analyses

For each probe, a grid representing the hybridization intensities of the 384 clones was constructed. The hybridization intensities were analyzed visually taking into account the surrounding background noise. The spots which were much more intense than the local background noise of the negative spots were considered to be positive, even if they were less intense than the most positive spots. These intermediate responses may be due to a partial mismatch of the probe (following the PCR steps) or alternatively to less efficient transfer of certain spots onto the filter. The ambiguities were removed by hybridizing another filter with the same probe.

The six 384-well grids were entered into Microsoft Excel tabulators and a statistical analysis was carried out with Excel macros written in Microsoft Visual Basic, and repeating the analytical steps as described in the description. The program first converts the hybridization signals into data of a parental type using a mask system with an XOR Boolean function, before the statistical analysis. The statistical analyses were carried out according to the steps detailed in the description.

Numeric simulations were produced using a generator of random numbers and probability calculation routines. The program can be adjusted to simulate all possible biases in the probability of finding one or other of the parental types for the sequence regions corresponding to each of the probes, and also all the possible “links” between adjacent or distant segments. A first set of parameters made it possible to modulate the relative probabilities of finding one or the other of the parental types for each sequence region probed. A second set of parameters made it possible to introduce one (or more) genetic link between two (or more) sequence fragments (corresponding to two or more probes).

The simulation and statistical analyses programs were used to generate grids corresponding to various situations of links between fragments. In all the tests, the results of the statistical analyses were in agreement with the parameters entered into the simulation program. The method of combining these simulation and analysis techniques was also used to determine the statistical fluctuations over the data by performing analyses of 10 repeated cycles of simulations and analyses for each set of parameters. The generator of random numbers was reinitialized between each simulation in order to make them independent events.

### Example 2

### Construction of an Expression Library by DNA Shuffling of the Same Family

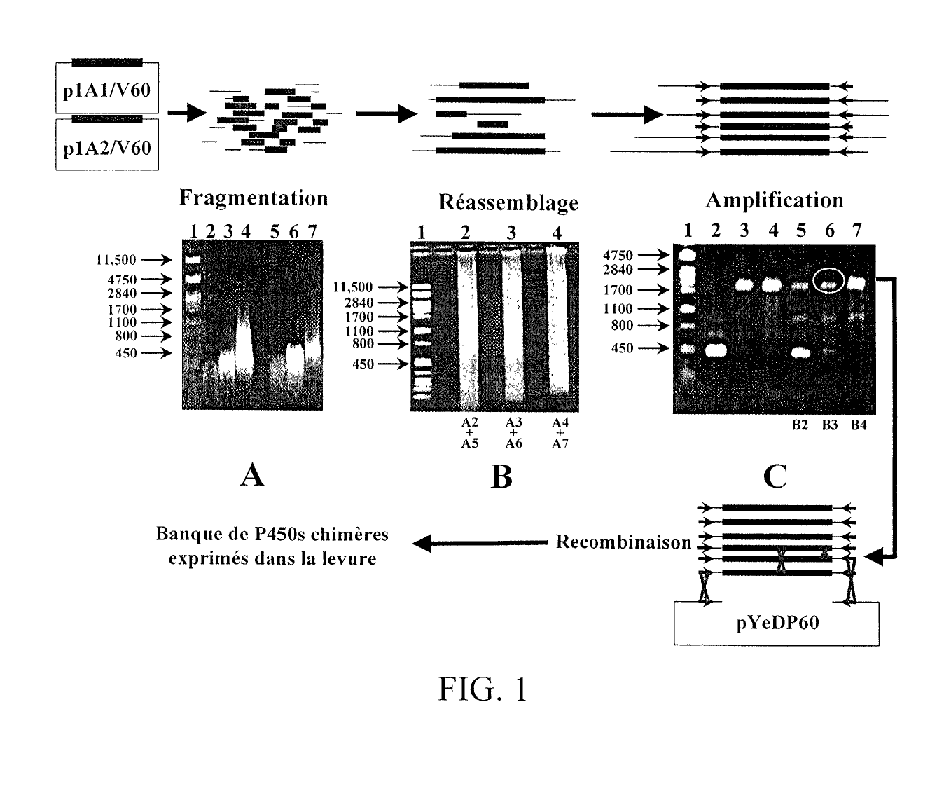

The principle of the strategy used is described in FIG. 1: it combines a step of in vitro DNA shuffling by modified PCR with a second step of in vivo shuffling by recombination in yeast. The latter step was also used as an effective cloning tool. This constitutes a complete shuffling strategy which allows expression in a eukaryotic cell and functional selection without the need for an intermediate cloning step in E. coli.

The first step (FIG. 1) consists of double-stranded fragmentation of the whole plasmid with DNAse I, producing DNA fragments which are small in size (FIG. 1A).

The results of the fragmentation of the plasmids p1A1/V60 and p1A2/V60 (FIG. 1A, lanes 2 and 5; 3 and 6; 4 and 7) were mixed in equimolar proportion and subjected to an original PCR program “gradual hybridizations” (see Example 1) involving 9 steps of hybridization ranging from 61° C. to 41° C. so as to force the recombination between fragments with little homology. As shown on FIG. 1B, in such situations, a large smear of high molecular weight DNA was formed whatever the fragments taken at the start.

Although this material was found to have properties of direct transformation of the yeast due to recombination between fragments in vivo and to the reconstitution of complete and functional yeast vectors (11 kb) (results not shown), a further PCR step, using primers located on the flanking CYC1 transcription initiation cDNA sequences and the PGK transcription termination sequences, was necessary in order to obtain a library of reasonable size (FIG. 1C, lanes 5, 6 and 7). The latter step resulted in amplification of a well-defined DNA band of approximately 1.9 kb comprising the “shuffled” cDNA and the flanking regions from the vector.

The PCR product shown in FIG. 1C, lane 6 was used to cotransform the yeast with pYeDP60 linearized at the expression site so as to use the homologous recombination properties (gap repair) of the yeast.

The cotransformation, into the yeast, of the good-sized cDNA library and of the linearized vector led to a series of recombination events which had already been observed in previous homeologous recombination, or gap-repair, experiments (37, 38, 43). The selection was based solely on the recircularization of the vector after one or more recombination events. The experiments gave approximately 10 000 clones.

Most of the yeast clones were transformed with several plasmids. Specifically, a heterogeneous population of plasmids was observed after extraction of DNA from a single yeast colony, transformation of E. coli and segregation of the clones.

This makes it possible to evaluate the complexity of the initial library at between 25 000 and 100 000 mosaic structures for a single yeast transformation experiment. The library can be used without modification for the functional selection.

Similar experiments using DNA fragments of lower molecular weights (less than 100 bp) as described in FIG. 1A, lanes 1 and 5 also produced a library which could be exploited, but less effectively. The higher molecular weight DNAs (FIG. 1A, lanes 4 and 7) were not used for constructing a library because of the possibilities of a high degree of contamination with parental structures.

### Example 3

### Statistical Analysis of a Subpopulation of the Library

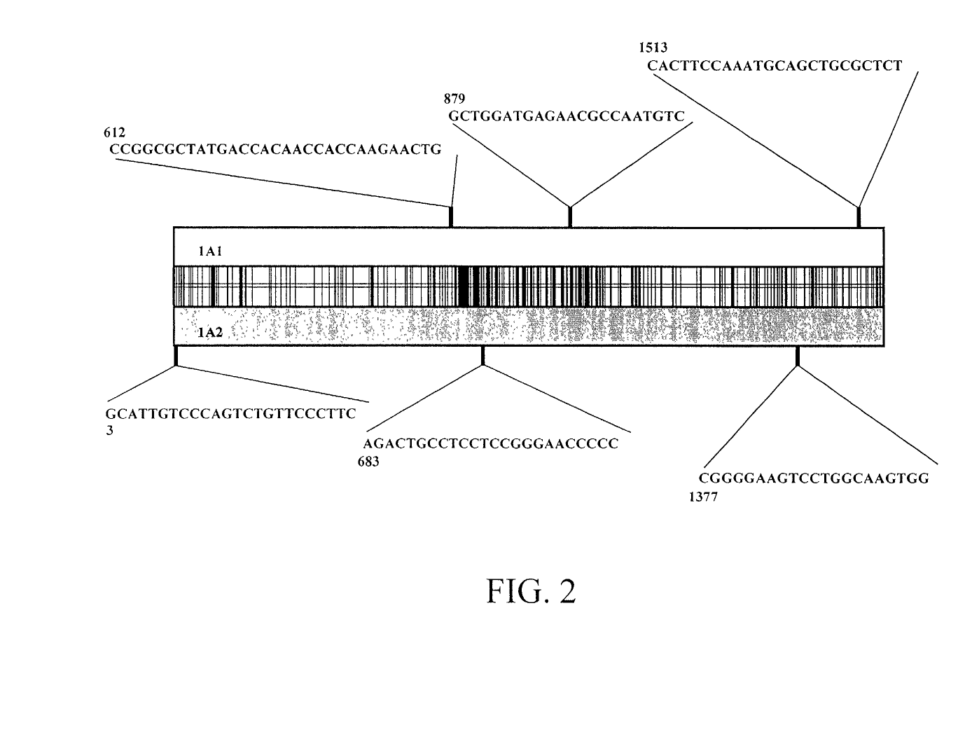

The plasmid DNA was prepared from the yeast library and used to transform E. coli using the ampicillin resistance marker present on the yeast plasmid. This step made it possible to segregate the individual plasmids which were initially present as a heterogeneous population in each yeast colony. A matrix was constructed using a 384-well microtitration plate containing 378 E. coli clones chosen randomly for structural analyses using 6 probes distributed along the sequence of the parental P450s described in FIG. 2 (SEQ ID No. 3 to SEQ ID No. 8). The remaining wells were seeded with bacteria transformed beforehand with control plasmids containing one or other of the parental sequences (P450 1A1 or 1A2).

The six probes (22-36 bases) were chosen so as to hybridize alternatively on the two parental sequences in regions of poor sequence similarity between the two parental P450s: 3 probes belonged to p1A1/V60 and 3 to p1A2/V60. Each probe was labeled with ³²P and used to hybridize the replicas on filters (under conditions promoting specific hybridizations). The experiments were repeated using various combinations of filters and probes in order to eliminate possible artifacts. The hybridization intensities were analyzed manually. The intermediate levels of hybridization intensity (about 15% of the spots) were considered to be positive responses. These responses must correspond to one-base-pair mismatches due to mutations induced by the various PCR steps (this being confirmed by the sequencing data (see later)) or to differences in efficiency of DNA transfer.

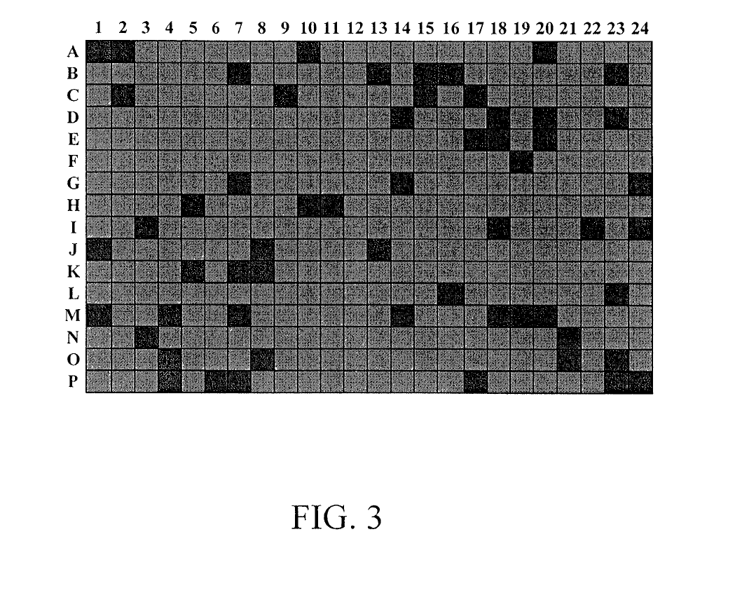

FIG. 3 shows the overall pattern of hybridization for the six probes. The frequency of structures having a hybridization pattern similar to one of the parents (hereinafter named “parentals”) for all the probes calculated in the library (FIG. 3A, dark squares) is 11.4% for structures corresponding to P450 1A2 and 2.4% for structures corresponding to P450 1A1. The sum of these two frequencies (13.8%) is greater than the theoretical value of 3.1% ((0.5)⁶+(0.5)⁶) corresponding to a totally random recombination of the parental sequence fragments. A “false-color” illustration of the various mosaic structures (not shown) clearly illustrates the excess of parental clones of 1A2 type or of 1A1 type, but suggests a quite homogeneous general distribution of the various types of mosaic structure.

With the aim of further characterizing the population, a statistical analysis was carried out using a program based on Excel tabulators and routines in Visual Basic. The probability of presence of each parental sequence at each of the 6 positions probed was calculated (Table 1). This frequency was quite homogeneous (0.56±0.02 for the fragments of 1A2 type) for the set of segments analyzed. The slightly higher frequency for the segments of 1A2 type probably reflects the error in the evaluation of the parental DNA contents during the mixing of the parental DNA fragments. The theoretical proportion of the parental sequences was recalculated with the new frequency values: 3.7% (0.58⁶+0.42⁶). The latter value still does not correspond to the proportion of parentals observed (13.8%).

TABLE 1
frequency of the portions of mosaic
sequences belonging to each parental type, at the
positions probed. The P1 to P6 probes begin at the
respective positions of the P450 1A1 or 1A2 sequences,
depending on the probe considered: 3, 612, 683, 1377
and 1513 (see figure 2). For each probe, the number of
hybridization signals relating to 1A1 or to 1A2 was
calculated and divided by the total number of clones
tested (378).

| | Frequency of | Frequency of |
|---|---|---|
| Probe | the type 1A1 | the type 1A2 |
| P1 | 0.48 | 0.52 |
| P2 | 0.43 | 0.57 |
| P3 | 0.45 | 0.55 |
| P4 | 0.45 | 0.55 |
| P5 | 0.44 | 0.56 |
| P6 | 0.41 | 0.59 |
| Mean ± S.D. | 0.43 ± 0.02 | 0.56 ± 0.02 |

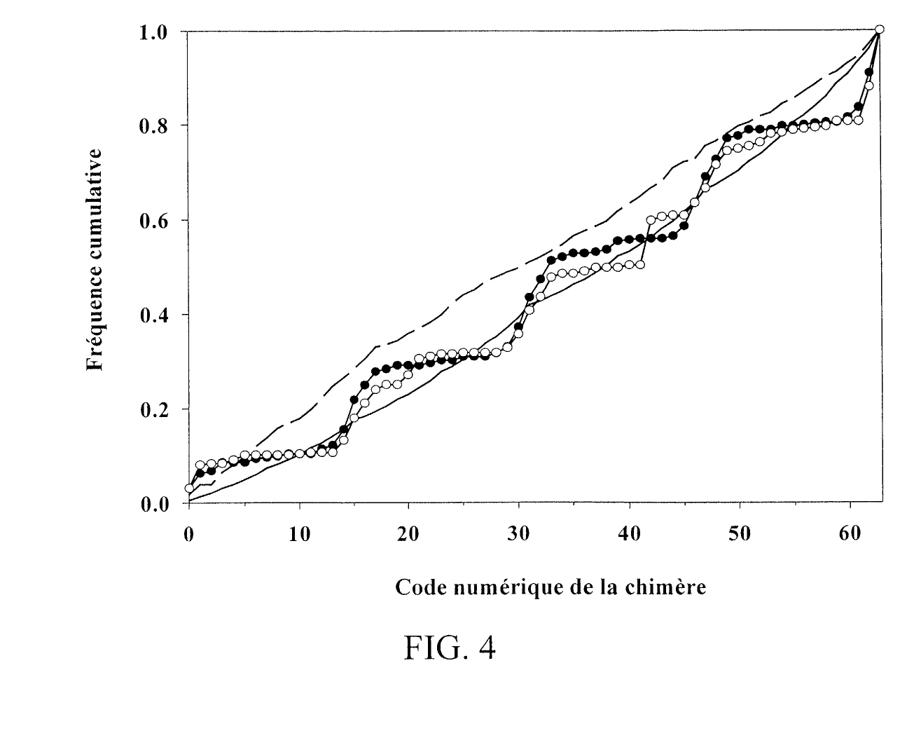

In order to characterize the population in greater detail, the curve of the cumulative frequencies for the probability of observing the 64 detectable classes of chimeras was calculated (FIG. 4). A binary code which arbitrarily associates a value of 0 or 1 depending on the nature of each segment (1A1 or 1A2), for segments 1 to 6, for each mosaic structure was used. The 1A1 and 1A2 parental sequences correspond to the codes 0 and 63, respectively. The experimental curve (FIG. 4, open circles) has an uneven appearance comprising five plateaus. The appearance of these plateaus was completely unexpected, and unpredictable, since they do not correspond to what would have been expected in the case of the recombination between the various fragments being independent.

Three theoretical curves were then calculated as described in Example 1 using approaches of the Monte Carlo type (numeric simulations), using various hypotheses:

(i) an equal probability of finding the various parental types at the sequence regions corresponding to the various probes, and total independence of the nature of each sequence segment;

(ii) hypothesis (i), but with a 55.8% probability of finding fragments of type 1A2 at the sequence regions corresponding to the various probes;

(iii) hypothesis (ii), but the probability of shuffling between the various sequence segments is no longer infinite (imperfect mixing), and with variable links between the nature of consecutive segments.

The cumulative frequency curve (FIG. 4) corresponding to hypothesis (i) is linear, whereas in the case corresponding to hypothesis (ii), the curve is rounded but remains even. This curve (which reflects the true percentage of parental fragments) effectively reproduces correctly the overall appearance of the curve calculated from the experimental results, but it does not show the plateaus observed.

Many curves corresponding to hypothesis (iii) were generated with various types of link between segments and a curve corresponding to the experimental curve was found (closed circles). The addition of suitable genetic links between the probed sequences makes it possible to determine a corresponding curve which follows the experimental curve. Of course, several solutions should be possible here, but a probability of link between parental fragments of 0.1; 0.6; 0.85; 0.1; 0.1 between the probed segments 1-2, 2-3, 3-4, 4-5 and 5-6, respectively, gives a satisfactory result. These results suggest that, even though the proportion of each parental type along the sequence is homogeneous, the probability of shuffling depends on the sequence segment considered. Thus, the plateaus of the results' curve obtained correspond to a correlation between various sequence segments.

The calculation of the frequencies of each parental type in the population was simulated after incorporating the link probabilities into the model. The mean results resulting from 10 computer simulations give a frequency of parental-type structures of 13.9±1.3% (of which 9.8±1.4% for 1A2 and 4.1±1.09% for 1A1), which corresponds quite well to the experimental values of 13.8% (11.4% for 1A2 and 2.4% for 1A1). The heterogeneity of the probability of shuffling along the sequence may therefore be entirely responsible for the apparent excess of parental-type structures in the population.

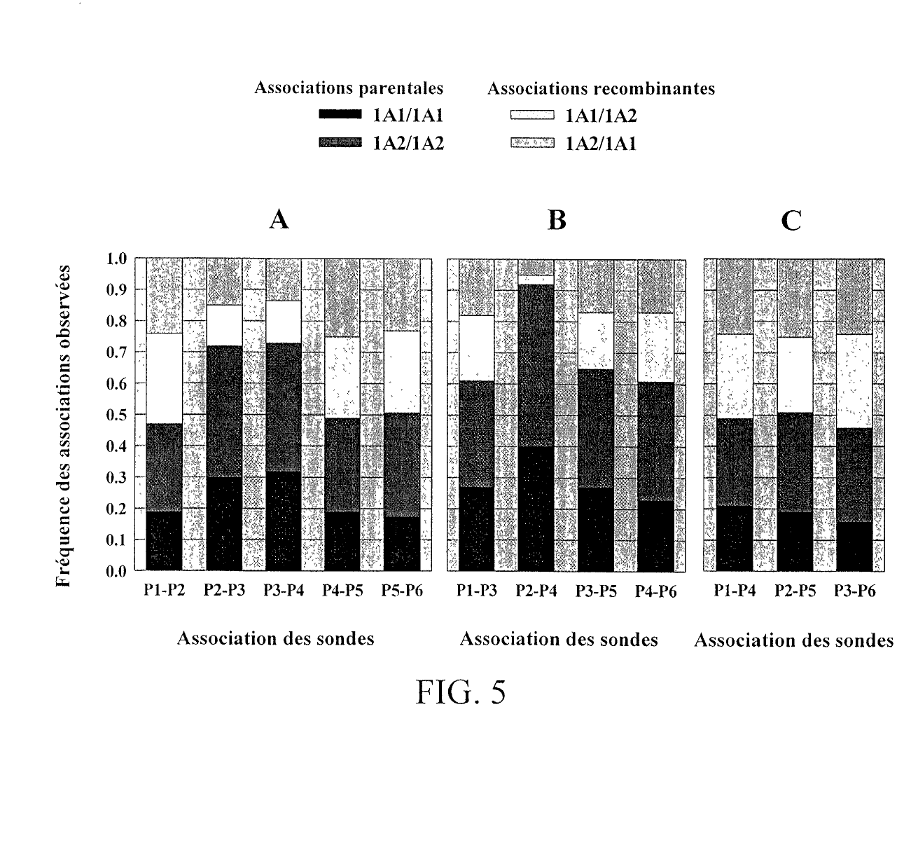

In order to verify the existence of links between fragments, the combinations between the various probes were analyzed. FIG. 5 shows the frequencies of the combinations of sequence regions of the same parental type and of different parental type for each one of the possible probe combinations.

In FIG. 5A, the probability of close combinations (between adjacent regions) can be seen. This clearly demonstrates that the P1-P2, P4-P5 and P5-P6 combinations show complete independence, unlike the P2-P3 and P3-P4 combinations which show a decrease in the frequency of combination between fragments of different parental type.

FIG. 5B shows the combination between two probes separated by a probe. Once again, a combination may be observed which shows an almost complete link between P2 and P4. The other combinations show the probes to be completely independent of one another.

This is also true for combinations between probes which are further apart (FIG. 5C). Other long distance combinations (P1-P5; P2-P6 and P1-P6) were calculated, reveal the same characteristics as those of FIG. 5C and are not shown herein.

These results clearly confirm the predictive model even though the number of links in the model is only 2. Surprisingly, the values obtained for these data do not correspond to a genetic model. Specifically, the distance (between the linked segments) appears to be greater in the case of P2-P4 compared to P2-P3 or P3-P4. A possible explanation for this phenomenon may be linked to the number of possible crossing-over events in this region (P2-P4).

The existence of plateaus corresponding to a correlation between fragments, when the analysis described above is used, makes it possible to draw an important conclusion. Specifically, when a functional selection pressure is exerted on the clones, it is probable that it will introduce a greater bias toward correlations between various regions of the genes studied. Thus, it may be possible to define patterns of combination between several regions of the gene, which are linked to functional properties and/or activities. This should make it possible to accelerate the process of defining proteins with enhanced functions and/or properties, by choosing the sequences to be combined.

### Example 4

### Selection of Functional Clones

A major advantage of the shuffling strategy developed in the present invention is that the library is, for the first time, directly constructed in a eukaryotic microorganism (yeast) . It is, in addition, possible to use yeast strains in which the genome has been modified so as to allow reconstitution of complex protein (enzymatic) systems.

In the experiments of the present invention, yeast strains with a modified genome were used, so as to allow the reconstitution of a membrane-bound system with coupling of the various partners. The transformed yeast clones resulting from the shuffling steps can then be used without further modification, for functional screening of the activity of the mosaic proteins constructed.

The use of the primary library also offers the advantage that it consists of clones containing multiple mosaic plasmids which considerably enhances the complexity of the library and makes it possible to screen the activities of several mosaic proteins by assaying the activity on just one yeast clone.

However, it is clear that the clones selected for their functionality require a further segregation step for a more detailed biochemical study. This segregation perhaps carried out by repeated subcloning or by extracting DNA from the positive clones, followed by transfer into E. coli and retransformation of yeast.

The following experiments demonstrate the feasibility of a direct functional selection in vivo in microtitration plates.

The method is based on a universal technique for detection by coloration of the aromatic phenols formed by direct in vivo bioconversion of aromatic polycyclic hydrocarbons in cultures in 96-well microplates (see Example 1).

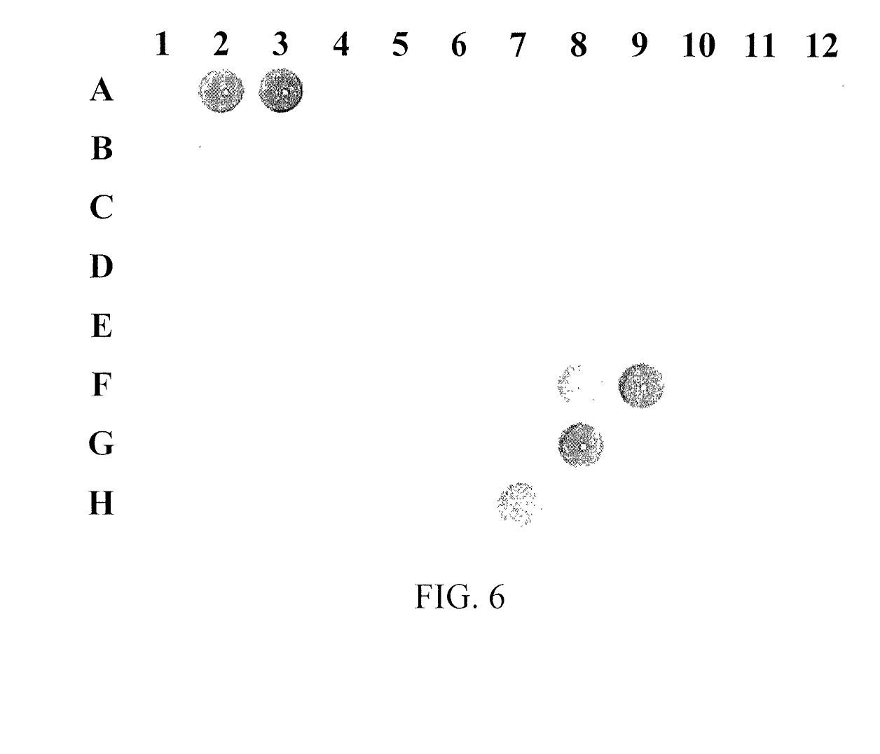

The phenol derivatives were then extracted via hydrophobic attachments (on C18 resins) directly on microplates and revealed by colorimetry subsequent to coupling with diazo-fast dye precursors (FIG. 6).

The 1A1/1A2 mosaic library was screened using naphthalene, which is a good substrate for the two parental enzymes. With the aim of determining the true proportion of functional structures, the primary library in yeast was transferred into E. coli and 96 independent (and therefore containing only one type of plasmid) clones were used to retransform the yeast in microtitration plates. The frequency of functional clones under such conditions (12% for the library constructed with Taq DNA polymerase) was reconfirmed by conventional methods using analyses of the extracted products by HPLC.

These controls made it possible to observe that the colorimetric detection is reliable and sufficiently sensitive to detect clones with a naphthalene hydroxylase activity representing only 10% of the parental activity (these differences in amounts of metabolites produced possibly being due to differences in activities but also in expression of the mosaic enzymes).

The detection method used also proved to be effective for the detection of metabolites derived from the metabolism of phenanthrene or of other aromatic polycyclic hydrocarbons.

### Example 5

### Library Sequence Analyses

Five clones selected randomly, independently of functional criteria, and the five clones chosen in the subpopulation of functional clones (see later for selection) were sequenced. These structures proved to be mosaics also containing additional mutations.

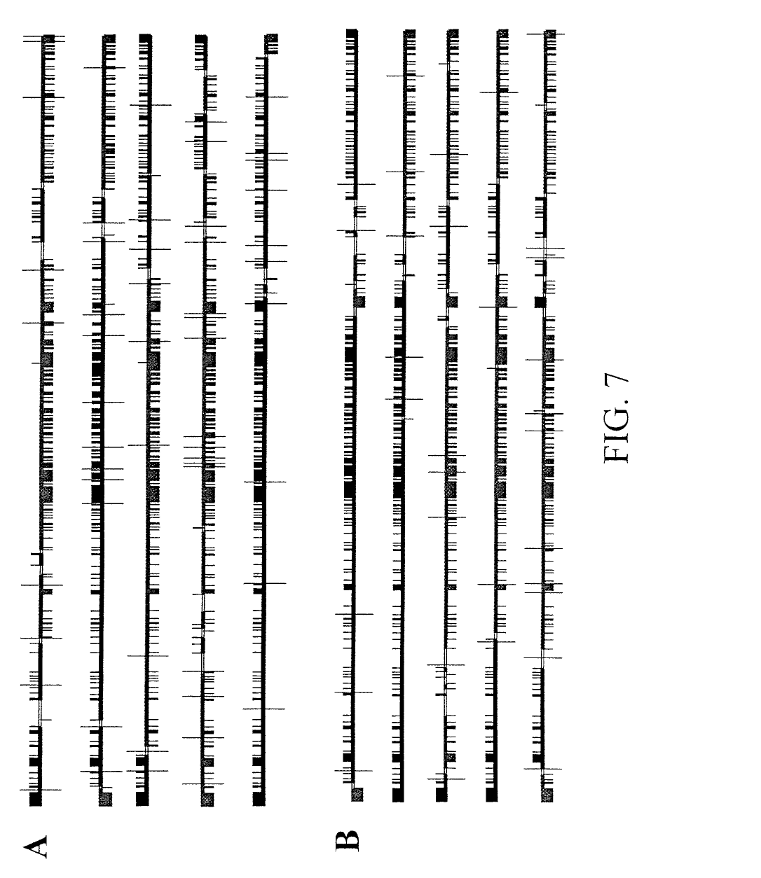

The mosaic structures are described in FIG. 7. The figure is based on an alignment between the mosaic structures and the two parental sequences, and was produced using suitable software: for each structure, a nucleotide alignment was produced with the two parental sequences. These alignments were used as starting data for a visualization program which generated the figure, illustrating the portions of sequences belonging to the 1A1 or 1A2 parental P450s in gray or in black, respectively, and adding upper or lower thin vertical lines to indicate the regions of nucleotide mismatch with the second parental structure. Furthermore, lines which cross the sequences indicate the positions of sequences which do not match any of the two parental sequences and which must therefore correspond to mutations. The software also illustrates transparent horizontal portions which correspond to segments of sequences for which it was not possible to determine whether they belonged to one or other of the parental types, by sequence analysis.

The analysis of these 10 randomly selected sequences confirms the presence of mosaic structures for each sequence. In analyzing all of these structures, a mean number of different fragments of 5.4±2.2 may be noted. The size distribution for these fragments is homogeneous. For the 54 fragments considered, 32 are between 0 and 200 bp in size, 12 are between 200 and 500 bp and 10 are between 500 and 1000 bp. In addition, approximately 60% of the fragments are less than 200 bp in size, the size of the smallest fragment exchanged being approximately 20 bp. These results are in agreement with the mean size of the starting fragments derived from the fragmentation with DNase I (200-300 bp, see FIG. 1A).

The analysis of the naphthalene hydroxylase activity of the 5 randomly chosen clones showed that only one was active (clone A1). It was subsequently considered to be an active clone, in the same way as the 5 chosen on activity criteria. The mean mutation rate per sequence was calculated for the active and inactive clones. For the inactive clones (A2, A3, A4 and A5), the mean number of mutations is 14.0 (±4.2). For the active clones, it is lower (8.3±3.2). This is not surprising, because of the method of selection (activity). In fact, the sequences of the inactive clones may contain early stop codons.

Finally, the various results observed in the statistical analyses were confirmed by the sequence data. In addition, even though the number of clones sequenced is low (10), the data obtained provide a detailed view of some mosaic structures. The link between fragments observed (between 2, 3 and 4) in the statistical analyses is also observed in these sequences. Specifically, no exchange of fragments is observed in the central portion corresponding to said probes.

The high mutation rate is in agreement with a relatively low proportion of functional structures (15%) in the population. However, similar shuffling experiments carried out using more reliable enzymes than Taq DNA polymerase, such as the Pfu or Dynazyme EXT DNA polymerase, gave a higher proportion (80-90%) of functional structures. The mutation rate may thus be adjusted as required.

The examples above illustrate one aspect of the invention, and those skilled in the art are able to make the adjustments required in order to generalize the teachings, without departing from the spirit of the invention.

### REFERENCES

1. van der Meer et al. (1992) Microbiological Reviews, 56(4), 677-94.

2. Stemmer, W. P. (1994) Nature, 370(6488), 389-91.

3. Stemmer, W. P. (1994) Proc. Natl. Acad. Sci. USA, 91(22), 10747-51.

4. Crameri et al. (1997) Nature Biotechnology, 15(5), 436-8.

5. Zhang et al. (1997) Proc. Natl. Acad. Sci. USA, 94(9), 4504-9.

6. Crameri et al (1996) Nature Biotechnology, 14(3), 315-9.

7. Crameri et al (1996) Nature Medicine, 2(1), 100-2.

8. Giver and Arnold (1998) Current Opinion in Chemical Biology, 2(3), 335-8.

9. Giver et al (1998) Proc. Natl. Acad. Sci. USA, 95(22), 12809-13.

10. Kumamaru et al (1998) Nature Biotechnology, 16(7), 663-6.

11. Moore et al (1997) J. Mol. Biol., 272(3), 336-47.

12. Moore and Arnold (1996) Nature Biotechnology, 14(4), 458-67.

13. Yano et al (1998) Proc. Natl. Acad. Sci. USA, 95(10), 5511-5.

14. Harayama, S. (1998) Trends In Biotechnology, 16(2), 76-82.

15. Crameri et al (1998) Nature, 391(6664), 288-91.

16. Nixon et al (1998) Trends In Biotechnology, 16(6), 258-64.

17. Kimura et al (1997) Journal of Bacteriology, 179(12), 3936-43.

18. Back, K. and Chappell, J. (1996) Proc. Natl. Acad. Sci. USA, 93, 6841-5.

19. Campbell et al (1997) Nat Biotechnol, 15(5), 439-43.

20. Nelson et al. (1987) In Guenguerich, F. P. (ed.), Mammalian cytochrome P-450. CRC Press, Boca Raton and Florida.s, 1987, pp. 19-79.

21. Harris, C. C. (1989) Carcinogenesis, 10(9), 1563-6.

22. Kadlubar et al. In Guenguerich, F. P. (ed.), Mammalian cytochrome P-450. CRC Press: Boca Raton and Florida.s., 1987, pp. 81-130.

23. Buters et al. (1999) Drug Metab Rev, 31(2), 437-47.

24. Kawajiri et al. (1990) Princess Takamatsu Symposia, 21, 55-61.

25. Kawajiri et al. (1990) FEBS Letters, 263(1), 131-3.

26. Kawajiri et al. (1993) Critical Reviews in Oncology-Hematology, 14, 77-87.

27. Mace et al. (1994) Molecular Carcinogenesis, 11(2), 65-73.

28. Joo et al. (1999) Chemistry & Biology, 6(10), 699-706.

29. Shao and Arnold (1996) Current Opinion in Structural Biology, 6(4), 513-8.

30. Arnold, F. H. (1998) Nature Biotechnology, 16(7), 617-8.

31. Michnick, S. W. and Arnold, F. H. (1999) Nat Biotechnol, 17(12), 1159-60.

32. Kikuchi et al. (1999) Gene, 236(1), 159-67.

33. Kikuchi et al. (2000) Gene, 243(1-2), 133-7.

34. Ostermeier et al. (1999) Nat Biotechnol, 17(12), 1205-9.

35. Volkov et al. (1999) Nucleic Acids Res, 27(18), e18.

36. Okuta et al (1998) Gene, 212(2), 221-8.

37. Pompon, D. and Nicolas, A. (1989) Gene, 83(1), 15-24.

38. Mezard, C., Pompon, D. and Nicolas, A. (1992) Cell, 70(4), 659-70.

39. Cullin, C. and Pompon, D. (1988) Gene, 65(2), 203-17.

40. Truan et al (1993) Gene, 125(1), 49-55.

41. Pompon et al. (1997) J Hepatol, 26 Suppl 2, 81-5.

42. Urban et al. (1990) Biochimie, 72(6-7), 463-72.

43. Bellamine et al. (1994) Eur J Biochem, 225(3), 1005-13.

44. Sambrook et al. (1989) Molecular cloning: a laboratory manual. 2nd Ed. Cold Spring Harbor Lab., Cold Spring Harbor, N.Y.

45. Lorimer, I. A. and Pastan, I. (1995) Nucleic Acids Res, 23(15), 3067-8.

46. Zhao, H. and Arnold, F. H. (1997) Nucleic Acids Research, 25(6), 1307-8.

47. Pompon et al. (1996) Methods Enzymol, 272, 51-64.

48. Pompon, D. (1988) Eur J Biochem, 177(2), 285-93.

49. Smith and Waterman (1981) Ad. App. Math. 2: 482

50. Neddleman and Wunsch (1970) J. Mol. Biol. 48: 443

51. Pearson and Lipman (1988) Proc. Natl. Acad. Sci. USA 85: 2444

1. Method for constructing a combinatorial functional expression library using a combinatorial library of nucleic acids belonging to the same gene family, characterized in that it comprises the steps consisting in:
a. introducing said combinatorial library of nucleic acids into a yeast, simultaneously with an expression vector,
b. obtaining said functional expression library by
homologous recombination of said combinatorial library of nucleic acids with said expression vector in said yeast, and
homologous or homeologous (between similar but not identical sequences) recombination, between the various nucleic acids of the combinatorial library introduced into said yeast, in order to increase the complexity and diversity of the combinatorial functional expression library obtained.

2. Method according to claim 1, characterized in that said combinatorial nucleic acid library is a mixture of PCR products obtained by amplifying a combinatorial open reading frame library, using a pair of primers located in regions flanking said open reading frames, said combinatorial library being obtained from homologous or sequence variant DNAs differing by one or more mutations.

3. Method according to claim 2, characterized in that said regions flanking the open reading frames are promoter and terminator regions which allow expression in yeast.

4. Method according to either of claims 2 and 3, characterized in that said combinatorial open reading frame library is obtained by reassembly by “primer extension” of fragmentation products from at least two open reading frames encoding functional proteins, said open reading frames exhibiting more than 40% sequence identity with one another.

5. Method according to claim 4, characterized in that said step of reassembly by “primer extension” is carried out by PCR.

6. Method according to claim 5, characterized in that each cycle of said step of reassembly by PCR has at least four hybridization stages of more than 60 seconds, with decreasing temperatures regularly spaced out.

7. Method according to one of claims 4 to 6, characterized in that said fragmentation products are obtained using an autonomous yeast expression vector more than 7 kb in total size (including the open reading frames).

8. Method according to claim 7, characterized in that said expression vector is an expression vector for a eukaryotic cell, and a shuttle for yeast.

9. Method according to either of claims 7 and 8, characterized in that said expression vector also contains the elements required for autonomous replication in Escherichia coli.

10. Method according to one of claims 7 to 9, characterized in that said expression vector contains an open reading frame encoding a eukaryotic membrane-bound enzyme.

11. Method according to claim 10, characterized in that said eukaryotic enzyme is chosen from the group consisting of eukaryotic cytochromes P450, eukaryotic conjungation enzymes (phase II enzymes) and members of the eukaryotic ABC transporter family.

12. Method according to one of claims 1 to 11, characterized in that said expression vector with which the recombination is carried out in the yeast is linearized at the normal cDNA cloning site and has transcription promoter and termination sequences, the recombination being carried out at the level of said sequences.

13. Method according to one of claims 1 to 12, characterized in that said expression vector also has the capacity to replicate autonomously in eukaryotic cells and/or in Escherichia coli.

14. Method according to one of claims 1 to 13, characterized in that the yeast strain used has a genetic modification allowing the overexpression of at least one protein chosen from the group consisting of an endogenous or exogenous P450 reductase, an adrenodoxin, an adrenodoxin reductase, a heterologous cytochrome b5 and a phase II enzyme (in particular an epoxide hydrolase).

15. Method according to one of claims 1 to 14, characterized in that step b. is followed by the steps consisting in:
a. extracting the plasmid DNA from at least one yeast clone,
b. transforming an Escherichia coli strain with said extracted plasmid DNA and selecting the transformed clones on suitable medium in order to discriminate between the elements of the combinatorial functional expression library.

16. Combinatorial functional expression library obtained using a method according to one of claims 1 to 15.

17. Method for producing functional active mosaic proteins, characterized in that a combinatorial functional expression library is constructed using a method according to one of claims 1 to 15, in that the mosaic proteins are expressed and in that the functional active mosaic proteins are selected by studying their activity.

18. Method according to claim 17, characterized in that said functional active mosaic proteins are derived from enzymes.

19. Method according to claim 18, characterized in that said functional active mosaic proteins are derived from cytochrome P450s.

20. Functional active mosaic protein obtained using a method according to one of claims 17 to 19.

21. Method for analyzing a combinatorial functional expression library according to claim 16, characterized in that it comprises the following steps:
a. transformation of an Escherichia coli strain with the plasmid DNA extracted from the yeast strain or from a pool of yeasts,
b. hybridization of the plasmid DNA contained in each of the individual Escherichia coli clones obtained at the end of step a. with one or more probe(s) specific for a parental sequence.

22. Method according to claim 21, characterized in that said hybridization takes place on a DNA macro- or microarray, said array consisting either of the plasmid DNA contained in each of the individual Escherichia coli clones obtained at the end of step a., or of a PCR product thereof, or of said specific probes, attached to a solid support, each of the nucleic acids being located via its position in said array.

23. Method for determining links between sequence signatures and functional signatures of a protein, characterized in that it comprises the steps consisting in
a. preparing a combinatorial functional expression library using a method according to one of claims 1 to 15,
b. producing the functional active mosaic proteins using a method according to one of claims 17 to 19,
c. analyzing the functional differences and/or the differences in activity between said mosaic proteins,
d. analyzing the nucleic acids corresponding to said mosaic proteins using a method according to claim 21 or 22, optionally followed by a method for analyzing a hybridization footprint, comprising the steps consisting in:
i. calculating the frequency of appearance of each of the possible combinations,
ii. defining a signature of the statistical distribution of the combinations, using suitable mathematical and statistical processing
e. relating the differences in sequence structures observed in step d. with the functional differences and/or the differences in activity observed in step c.

24. Method for predicting structures which have a given function, characterized in that the method according to claim 23 is implemented in order to identify the sequence regions, or the links between the sequence regions, related to said function, and in that the structure being sought is deduced therefrom.

25. Method for obtaining a protein having enhanced properties, characterized in that it comprises the steps consisting in:
a. constructing a combinatorial functional expression library using a method according to one of claims 1 to 15,
b. analyzing said combinatorial functional expression library using a method according to either of claims 21 and 22,
c. analyzing the hybridization footprints obtained in step b. using a method for analyzing hybridization footprints, comprising the steps consisting in:
i. calculating the frequency of appearance of each of the possible combinations,
ii. defining a signature of the statistical distribution of the combinations, using suitable mathematical and statistical processing
d. determining the links between the sequence structures and functional structures of the proteins by comparing said hybridization footprints with the properties of the corresponding mosaic proteins, using a method according to claim 23,
e. predicting the structures of interest or the structural organizations in the mosaic proteins using a method according to claim 24,
f. repeating steps a. to e., using, as starting nucleic acids for generating the combinatorial functional expression library, the nucleic acids bearing the structures of interest or the structural organizations identified in step e., a sufficient number of times to obtain the protein having desired enhanced properties.

26. Protein obtained using the method according to claim 25.

27. Method for determining a protein structure which is important in response to a selection pressure, using a combinatorial functional expression library which has been obtained using a method according to the invention, for the elements of which a signature has been obtained, comprising the steps of:
normalizing said library, by making the signatures homogeneous,
applying a selection pressure,
analyzing the sequence signature of the novel library thus obtained, compared to the normalized starting library,
studying the change in the signature of the novel library obtained, compared to the normalized starting library, thus deducing the structures which are present or absent in response to the selection pressure.
